(19) United States
(12) Patent Application Publication
(10) Pub. No.: US 2011/0104212 A1
(43) Pub. Date: May 5, 2011

(54) POLYMERIC COMPOSITIONS WITH ENHANCED SALINE HOLDING CAPACITY AND THEIR METHOD OF PREPARATION AND USE

(75) Inventors: Alan Strickland, Lake Jackson, TX (US); George Grass, Tahoe City, CA (US)

(73) Assignee: SORBENT THERAPEUTICS, INC., Vernon Hills, IL (US)

(21) Appl. No.: 12/675,689

(22) PCT Filed: Aug. 29, 2008
(86) PCT No.: PCT/US08/74847

§ 371 Date: Jan. 18, 2011

Related U.S. Application Data
(60) Provisional application No. 60/968,818, filed on Aug. 29, 2007.
(60) Provisional application No. 60/968,820, filed on Aug. 29, 2007.
(60) Provisional application No. 60/968,821, filed on Aug. 29, 2007.

(51) Int. Cl. A61K 9/00; A61K 31/78; B01J 39/20

(52) U.S. Cl. 424/400; 424/78.1; 521/38

(57) ABSTRACT

Cross-linked polyelectrolyte polymers that absorb 60-fold or more, including greater than 60-fold, greater than 70-fold, greater than 80-fold, greater than 90-fold, greater than 100-fold, or greater than 110-fold or more, of their mass in a saline solution are disclosed. Methods for preparing such polymers with enhanced saline holding capacity and methods for treating diseases, disorders or conditions involving fluid overload and/or ion imbalances by administering the polymers are disclosed.

### FIELD OF THE DISCLOSURE

The present disclosure relates generally to polymeric compositions with enhanced saline holding capacity. More specifically, cross-linked polyelectrolyte polymers that absorb about 60-fold or more, including greater than 60-fold, 70-fold, 80-fold, 90-fold, 100-fold, 110-fold or more, of their mass in a saline solution are disclosed. The present disclosure also relates generally to methods for preparing such polymers with enhanced saline holding capacity. The present disclosure also relates generally to methods for treating diseases, disorders or conditions involving fluid overload and/or ion imbalances by administering one or more polymeric compositions of the present disclosure.

### BACKGROUND

Numerous diseases and disorders are associated increased retention of fluid (e.g., congestive heart failure and end stage renal disease (ESRD) and chronic kidney disease (CKD)) and/or with ion imbalances (e.g., hyperkalemia, hypercalcemia, hyperphosphatemia and hyperoxalemia). For example, patients affflicted with retention of fluid often suffer from edema (e.g. pulmonary edema and/or edema of the legs) and the buildup of waste products in the blood (e.g., urea, creatinine, other nitrogenous waste products, and electrolytes or minerals, such as sodium, phosphate and potassium). Additionally, patients afflicted with an increased level of potassium may exhibit a variety of symptoms ranging from malaise, palpitations, muscle weakness and in severe cases, cardiac arrhythmias. Also, for example, patients afflicted with increased levels of sodium (e.g., hypernatremia) may exhibit a variety of symptoms including, lethargy, weakness, irritability, edema and in severe cases, seizures and coma.

Treatments for diseases or disorders associated with an increased retention of fluid (e.g., fluid overload) and/or ion imbalances attempt to decrease the retention of fluid and restore the ion balance. For example, treatment of diseases or disorders associated with ion imbalances may employ the use of ion exchange resins to restore ion balance. Treatment of diseases or disorders associated with an increased retention of fluid may involve the use of diuretics (e.g., administration of diuretic agents) and/or dialysis, such as hemodialysis or peritoneal dialysis and remediation of waste products that accumulate in the body. Additionally or alternatively, treatment for ion imbalances and/or increased retention of fluid may include restrictions on dietary consumption of electrolytes and water. However, the effectiveness and/or patient compliance with present treatments is less than desired.

### SUMMARY

Cross-linked polyelectrolyte polymers that absorb about 60-fold or more of their mass in aqueous saline solution are disclosed. Polymers that can absorb more than 60-fold, including at least about 70, 80, 90, 100 or 110-fold, their mass in aqueous saline solution are also disclosed. The polymers can be substantially free of soluble polymer and can comprise a variety of concentrations of bound counterions, including cations such as sodium. The polymer can be formed into spherical or nearly spherical particles (e.g., beads) which can be disrupted by processes such as grinding or milling (e.g., disrupted beads). These polymer particles may be encapsulated in a capsule. These polymer particles or capsules containing the particles can be coated with a coating such as an enteric coating. The coating can be complete or substantially complete such that particles can pass directly into the intestine before becoming exposed for fluid absorption.

Methods for treating a patient having a fluid overload condition are also disclosed. The methods involve identifying a patient having a fluid overload condition, obtaining a cross-linked polyelectrolyte polymer that absorbs about 60-fold or more of its mass in saline and administering the cross-linked polyacrylic acid polymer to the intestine of the patient. The cross-linked polymer can be in a particle form, including where the particles are encapsulated, and can have any of the aforementioned characteristics. The methods can include directly administering the particles to the jejunum.

Methods for preparing the aforementioned polymers are also disclosed. The methods can include obtaining a cross-linked polyelectrolyte in a spherical or nearly spherical particle form. The particles can be prepared in suspension polymerization with a cross-linker relatively insoluble in the solvent in which the monomer is dissolved (e.g., inverse suspension polymerization). The particles can be disrupted, collected and rinsed to remove soluble polymer. The rinsed particles can then be dried until they can absorb about 70 times their mass in an aqueous solution such as a neutral 0.154 M saline solution. In an embodiment, the particles can be about 700 microns or above prior to disruption.

Additional features and advantages are described herein, and will be apparent from, the following Detailed Description.

### DETAILED DESCRIPTION

The present disclosure provides cross-linked polyelectrolyte polymers, including compositions comprising cross-linked polyelectrolyte polymeric particles, such as disrupted cross-linked polyacrylate polymeric particles, with enhanced saline holding capacity. The cross-linked polyelectrolyte polymeric beads may absorb 60-fold or more, including greater than 60-fold, 70-fold, 80-fold, 90-fold, 100-fold or 110-fold or more, of their mass in aqueous saline solution. Surprisingly, it has been discovered that either (1) disruption of cross-linked polyelectrolyte polymeric beads, including cross-linked polyacrylate polymeric beads, into smaller particles (e.g., by milling or crushing) and washing the disrupted beads with purified water, or (2) placing cross-linked polyelectrolyte polymeric beads, including cross-linked polyacrylate polymeric beads, into purified water and agitating the beads, the residual soluble polymer in the polymeric beads may be reduced or eliminated and the saline holding capacity of the dried polymeric beads may be increased. Most surprisingly, disruption of the polyelectrolyte compositions with subsequent washing led to an unexpected increase in saline holding capacity of the polyelectrolyte composition. For example, when about 80% neutralized lightly crosslinked sodium polyacrylate beads are formed with particle sizes in the range of about 700 to about 1200 microns, a saline holding capacity of about 60 grams per gram of intact beads can be obtained. However, when such beads are disrupted, washed and dried as described above they have a surprisingly high saline holding capacity of about 90 grams per gram, for example, about 92 grams per gram at four hours and 110 grams per gram after overnight saline uptake.

Without wishing to be bound by any particular theory, it is believed that during manufacture of cross-linked polyelectrolyte polymeric beads (e.g., by inverse suspension polymerization of acrylic acid), a cross-linker (such as trimethylolpropane triacrylate) that is quite insoluble in water only slowly diffuses into the water droplet along with the free monomer (e.g., 20% polyacrylate) that is still present during the initial stages of the polymerization reaction. This could potentially create a concentration gradient of cross-linker across the radius of the bead and allow a series of different degrees of cross-linking to form. When the beads are disrupted into particles, including hemispheres and particles with a surface component, a more lightly cross-linked polymer may be exposed. This is quite unlike what would be expected from a preparation made in aqueous solutions with polymerizations performed with a cross-linker that is soluble in the aqueous phase (e.g., without a concentration gradient of cross-linker) or with diameters of the bead-like particles that are small enough to prevent establishment of a significant gradient of cross-linker in the droplet. Those skilled in the art will easily recognize the adaptation of the specific details above to the preparation of other superabsorbent polyelectrolyte polymers and the disruption of bead-like materials with washing to remove soluble polymer.

The present disclosure provides cross-linked polyelectrolyte polymers that absorb about 60-fold or more, including greater than 60-fold, 70-fold, 80-fold, 90-fold, 100-fold, 110-fold or more, of their mass in a saline solution.

In some embodiments, the cross-linked polyelectrolyte polymer is substantially free of soluble polymer.

In some embodiments, the cross-linked polyelectrolyte polymer is disrupted. In some embodiments, the polymer is substantially in the shape of a disrupted sphere or ellipsoid (e.g., disrupted beads). In some embodiments, the disrupted sphere or ellipsoid has a size of about 210 to 500 microns.

In some embodiments, the cross-linked polyelectrolyte polymer is polyacrylate.

In some embodiments, the cross-linked polyelectrolyte polymer comprises bound counterions. In some embodiments, the cross-linked polyelectrolyte polymer comprises cations. In some embodiments, the cross-linked polyelectrolyte polymer comprises one or more bound inorganic counterions. In some embodiments, the inorganic counterion is selected from the group consisting of: hydrogen, sodium, potassium, calcium, magnesium and ammonium. In some embodiments, the cross-linked polyelectrolyte polymer comprises one or more bound organic counterions. In some embodiments, the organic counterion is selected from the group consisting of: choline, arginine and lysine. In some embodiments, the cross-linked polyelectrolyte polymer comprises one or more inorganic counterions and at least one or more organic counterions.

In some embodiments, the cross-linked polyelectrolyte polymer is substantially coated. In some embodiments, the particles are substantially coated. In some embodiments, the particles are surrounded by a capsule. In some embodiments, the capsule is coated with a coating. In some embodiments, the coating is an enteric or delayed release coating.

Pharmaceutical compositions are also provided that comprise the cross-linked polyelectrolyte polymers of the present disclosure.

Methods of removing fluid from a subject are provided that comprise administering a cross-linked polyelectrolyte polymer of the present disclosure to the subject in an amount effective to remove fluid from the subject. Optionally, the methods may further comprise identifying a subject in need of removal of the fluid.

In some embodiments, the methods may further comprise administering to the subject one or more agents that increase the amount of fluid in the intestine. In some embodiments, the agent is selected from the group consisting of: non-absorbed saccharides (e.g., mannitol or sorbitol), water-soluble glycols (e.g., polyethylene glycol or polypropylene), and lubiprostone. In some embodiments, polyethylene glycol has a molecular weight between 400 and 10,000 Daltons. In some embodiments, the polyethylene glycol has a molecular weight between 400 and 4000 Daltons.

In some embodiments, the polymer is directly administered to the colon. In some embodiments, the polymer is directly administered to the small intestine. In some embodiments, the polymer is directly administered to the jejunum.

In some embodiments, the polymer is administered orally.

In some embodiments, the subject has cardiac disease. In some embodiments, the cardiac disease is congestive heart failure. In some embodiments, the subject has kidney disease. In some embodiments, the kidney disease is nephrosis, nephritis, chronic kidney disease (CKD), or end stage renal disease (ESRD). In some embodiments, the subject has an intestinal or nutritional disorder. In some embodiments, the nutritional disorder is kwashiorkor or gluten-sensitive enteropathy. In some embodiments, the subject has hepatic disease. In some embodiments, the hepatic disease is cirrhosis of the liver. In some embodiments, the subject has an endocrine, neurological or immune system disorder. In some embodiments, the endocrine disorder is preclampsia or eclampsia. In some embodiments, the neurological disorder is angioneurotic edema.

Methods of removing one or more waste products from a subject are provided that comprise administering a cross-linked polyelectrolyte polymer of the present disclosure to the subject in an amount effective to remove one or more waste products from the subject. Optionally, the methods may further comprise identifying a subject in need of removal of one or more waste products.

In some embodiments, the waste product is a metabolic waste. In some embodiments, the metabolic waste is urea, uric acid, creatinine, sodium or potassium.

In some embodiments, the methods may further comprise administering to the subject one or more agents that increase the amount of fluid in the intestine. In some embodiments, the agent is selected from the group consisting of: mannitol, polyethylene glycol and lubiprostone.

In some embodiments, the polymer is directly administered to the colon. In some embodiments, the polymer is directly administered to the small intestine. In some embodiments, the polymer is directly administered to the jejunum.

In some embodiments, the polymer is administered orally.

Methods are also provided for preparing cross-linked polyelectrolyte polymeric particles capable of absorbing about 60-fold or more, including greater than 60-fold, 70-fold, 80-fold, 90-fold, 100-fold or 110-fold or more, times its mass of an aqueous saline solution by (a.) obtaining cross-linked polyelectrolyte polymer in a spherical or nearly spherical form, (b.) disrupting the polymer in particles, (c.) washing the particles (e.g., to reduce residual soluble polymer content in the bead) and (d) drying the washed particles to obtain dry particles. The polymer of step (a.) may be in the form of beads. In some embodiments, the beads are greater than 500 microns, such as 500-1000 microns, 710-1000 microns or 500-710 microns.

In some embodiments, the methods may further comprise drying the washed particles.

In some embodiments, the cross-linked polyelectrolyte polymer is disrupted by milling. In some embodiments, the cross-linked polyelectrolyte polymer is disrupted by incubating in aqueous solution with agitation (e.g., stirring in water).

In some embodiments, the particles are washed with deionized water, distilled water or alcohol.

In some embodiments, the cross-linked polyelectrolyte polymer is disrupted in the dry state. In some embodiments, the cross-linked polyelectrolyte polymer is disrupted after swelling with purified water.

In some embodiments, soluble polymer content in the particles is reduced, including eliminated.

In some embodiments, the methods may further comprise substantially coating the dried, washed particles.

In some embodiments, the particles are surrounded by a capsule. In some embodiments, the capsule is coated with a coating. In some embodiments, the coating is an enteric or delayed release coating.

The present disclosure also provides cross-linked polyelectrolyte polymers prepared by the methods described herein.

### Preparation of Superabsorbent Polyelectrolyte Beads

Superabsorbent polyelectrolyte beads, including, for example, polyacrylate beads, may be prepared by methods known in the art, including by suspension methods (e.g., Buchholz, F. L. and Graham, A. T., “Modem Superabsorbent Polymer Technology,” John Wiley & Sons (1998)). Methods may include manufacture of polyelectrolyte beads by inverse suspension polymerization. Exemplary methods are provided below.

1. Manufacture of Superabsorbent Polyelectrolyte

Cross-linked polyelectrolyte polymers, including cross-linked polyelectrolyte polymeric beads, may be prepared by commonly known methods in the art. In an exemplary method, cross-linked polyelectrolyte polymers may be prepared as a suspension of drops of aqueous solution in a hydrocarbon (e.g., by inverse suspension polymerization).

Superabsorbent polyacrylates may be prepared by polymerization of partially neutralized acrylic acid in an aqueous environment where an appropriate cross-linker is present in small quantities. Given that there is an inverse relationship between the amount of fluid the superabsorbent polymer will absorb and the degree of cross-linking of the polymer, it desirable to have the minimum cross-linking possible to still produce a resin. However, there is also an inverse relationship between the degree of cross-linking and the percentage of polymer chains that do not cross-link and are therefore soluble polymer that does not contribute to the absorbency of the resin since it dissolves in the fluid. For example, superabsorbent polyacrylates may be designed to absorb about 35 times their mass in physiological saline as a compromise between maximal absorbency and minimal soluble polymer.

Since the amount of reactants used in an inverse suspension polymerization reaction varies depending upon the size of the reactor, the precise amount of each reactant used in the preparation of cross-linked polyelectrolyte polymer, such as polyacrylate may be determined by one of skill in the art. For example, in a five-hundred gallon reactor, about 190 to 200 pounds (roughly 85 to 90 kg) of acrylic acid may be used while in a three liter reactor 150 to 180 g of acrylic acid may be used. Accordingly, the amounts of each reactant used for the preparation of cross-linked polyacrylate are expressed as weight ratios to acrylic acid. Thus, acrylic acid weight is taken as 1.0000 and other compounds are presented in relation to this value. Exemplary amounts of reactants used for the preparation of cross-linked polyacrylate by an inverse suspension polymerization are presented in Table 1.

TABLE 1
Exemplary amounts of reactants in an inverse
suspension polymerization

| Substance | Low value | High Value |
|---|---|---|
| Acrylic acid | 1.0000 | 1.0000 |
| Water | 0.5000 | 3.0000 |
| Hydrophobic solvent | 1.2000 | 12.0000 |
| Base | 0.6600 (60% neutral) | 1.1100 |
| (expressed as 50% NaOH) | | (100% neutralized) |
| Crosslinker | 0.0030 | 0.0080 |
| Initiator | 0.0005 | 0.0200 |
| Chelating agent | 0.0000 | 0.0050 |
| Surfactant | 0.0050 | 0.0400 |

An exemplary inverse suspension reaction to form a superabsorbent polymer may involve preparation of two mixtures (e.g., a hydrophobic and an aqueous mixture) in two different vessels followed by combination of the mixtures to form a reaction mixture. One vessel may be designated as a hydrophobic compound vessel and the other may be designated as a aqueous solution vessel. The hydrophobic compounds may be mixed in a larger vessel that will become a reaction vessel, while an aqueous solution may be prepared in a smaller vessel that may be discharged into the reaction vessel.

A hydrophobic solvent may be introduced into the reaction vessel. As will be appreciated by one of skill in the art, a hydrophobic solvent (also referred to herein as the “oil phase”) may be chosen based upon one or more considerations, including, for example, the density and viscosity of the oil phase, the solubility of water in the oil phase, the partitioning of the neutralized and unneutralized ethylenically unsaturated monomers between the oil phase and the aqueous phase, the partitioning of the cross-linker and the initiator between the oil phase and the aqueous phase and/or the boiling point of the oil phase.

Hydrophobic solvents contemplated for use in the present disclosure include, for example, Isopar L, toluene, benzene, dodecane, cyclohexane, n-heptane and/or cumene. Preferably, Isopar L is chosen as a hydrophobic solvent due to its low viscosity, high boiling point and low solubility for neutralized monomers such as sodium acrylate and/or potassium acrylate. One of skill in the art will appreciate that a large enough volume of hydrophobic solvent is used to ensure that the aqueous phase is suspended as droplets in the oil rather than the reverse and that the aqueous phase droplets are sufficiently separated to prevent coalescence into large masses of aqueous phase.

One or more surfactants and one or more cross-linkers may be added to the oil phase. The oil phase may then be agitated and sparged with an inert gas, such as nitrogen or argon to remove oxygen from the oil phase. It will be appreciated that the amount of surfactant used in the reaction depends on the size of the desired beads and the agitator stir rate. This addition of surfactant is designed to coat the water droplets formed in the initial reaction mixture before the reaction starts. Higher amounts of surfactant and higher agitation rates produce smaller droplets with more total surface area. It will be understood by those of skill in the art that an appropriate choice of cross-linker and initiator may be used to prepare spherical to ellipsoid shaped beads. One of skill in the art will be capable of determining an appropriate cross-linker for the preparation of a specified cross-linked polyelectrolyte. For example, cross-linker choice depends on whether it needs to be hydrophobic or hydrophilic or whether it needs to resist acidic or basic external conditions. An amount of cross-linker depends on how much soluble polymer is permissible and how much saline holding capacity is needed.

Exemplary surfactants include hydrophobic agents that are solids at room temperature, including, for example, hydrophobic silicas (such as Aerosil or Perform-O-Sil) and glycolipids (such as polyethylene glycol distearate, polyethylene glycol dioleate, sorbitan monostearate, sorbitan monooleate or ocytl glucoside).

Cross-linking agents with two or more vinyl groups that are not in resonance with each other may be used, allowing for a wide variety in molecular weight, aqueous solubility and/or lipid solubility. Cross-linking agents contemplated for use in the present disclosure, include, for example, diethelyeneglycol diacrylate (diacryl glycerol), triallylamine, tetraallyloxyethane, allylmethacrylate, 1,1,1-trimethylolpropane triacrylate (TMPTA), and divinylbenzene.

An aqueous phase mixture may be prepared in another vessel (e.g., a vessel that is separate from that used to prepare the hydrophobic phase) by placing water into the vessel and adding a base to the water. It will be appreciated by one of skill in the art that the amount of base used in the vessel is determined by the degree of neutralization of the monomer desired. A degree of neutralization between 60% and 100% is preferred. Without wishing to be bound by a theory of the disclosure, it is believed that one-hundred percent neutralization minimizes the chance of suspension failure, but the highly charged monomer may not react as rapidly and may not pull hydrophobic cross-linkers into the beads. Considerations in choosing the degree of neutralization may be determined by one of skill in the art and include, for example, the effect of monomer charge (e.g., as determined by ionization of the cation from the neutralized molecules) on reaction rate, partitioning of the monomer and neutralized monomer between oil phase and aqueous phase and/or tendency to coalescence of the polymer chains during the reaction. The solubilities of sodium acrylate and sodium methacrylate in water are limited and are lower at lower temperatures (e.g., sodium acrylate is soluble at about 45% at 70° C. but less than 40% at 20° C.). This solubility may establish the lower limit of the amount of water needed in the neutralization step. The upper limit of the amount of water may be based on reactor size, amount of oil phase needed to reliably suspend the aqueous phase as droplets and/or the desired amount of polymer produced per batch.

Bases contemplated for use in the present disclosure include, for example, hydroxides, bicarbonates, or carbonates. Use of these bases allows neutralization of the acid monomer without residual anions left in the reaction mixture. It will be apparent to one of skill in the art that the cation used for the base may be chosen based on the planned use of the superabsorbent polymer. Normally, sodium bases are chosen since the superabsorbent polymers will be used in situations where saline solutions will be encountered. However, potassium bases, ammonium bases, and bases of other cations are contemplated for use in the present disclosure.

The water used in the reaction may be purified water or water from other sources such as city water or well water. If the water used is not purified water, chelating agents may be needed to control metals such as iron, calcium, and magnesium from destroying the initiator. Chelating agents contemplated for use with the present disclosure include, for example, Versenex 80. The amount of chelating agent added to the reaction mixture may be determined by one of skill in the art from a determination of the amount of metal in the water.

Once base is added to the water, the aqueous phase solution may be cooled to remove the heat released from dilution of the base and one or more classes of monomers may be added to react with the base. As will be appreciated by one of skill in the art, the monomers will be neutralized to the degree dictated by the amount of base in the reaction. The aqueous phase solution may be kept cool (e.g., below 35 to 40° C.) and preferably around 20° C. to prevent formation of prepolymer strands, dimers and/or possible premature polymerization.

Monomers are dissolved in water at concentrations of 20-40 wt % and polymerization may subsequently be initiated by free radicals in the aqueous phase. Monomers may be polymerized either in the acid form (pH 2-4) or as a partially neutralized salt (pH 5-7). The amount of water used to dissolve the monomer is minimally set so that all of the monomer (e.g., sodium acrylate) is dissolved in the water rather than crystallizing and maximally set so that there is the smallest volume of reaction mixture possible (to minimize the amount of distillation and allow the maximum yield per batch).

Exemplary monomer units contemplated for use in the present disclosure, include, for example, acrylic acid and its salts, methacrylic acid and its salts, crotonic acid and its salts, tiglinic acid and its salts, 2-methyl-2-butenoic acid (Z) and its salts, 3-butenoic acid (vinylacetic acid) and its salts, 1-cyclopentene carboxylic acid, and 2-cyclopentene carboxylic acid and their salts. Other cross-linked polyelectrolyte superabsorbent polymers may be based on sulfonic acids and their salts, phosphonic acids and their salts, or amines and their salts.

One or more initiators, free radical producers, may be added to the aqueous phase just before the aqueous phase is transferred into the oil phase. As will be appreciated by one of skill in the art, the initiator amounts and type used in the polymerization reaction depend on oil versus water solubility and the need for longer chain lengths. For example, a lower amount of initiator may be used in the polymerization reaction when longer chain lengths are desired.

In some embodiments, the initiator may be a thermally sensitive compound such as persulfates, 2,2′-azobis (2-amidino-propane)-dihydrocholoride, 2,2′-azobis (2-amidino-propane)-dihydrochloride and/or 2,2′-azobis (4-cyanopentanoic acid) persulfate or 2,2′-azobis(4-cyanopentanoic acid). Thermally sensitive initiators have the disadvantage that the polymerization does not begin until an elevated temperature is reached. For persulfates, this temperature is approximately 50 to 55° C. Since the reaction is highly exothermic, vigorous removal of the heat of reaction is required to prevent boiling of the aqueous phase. It is preferred that the reaction mixture be maintained at approximately 65° C. As will be appreciated by one of skill in the art, thermal initiators have the advantage of allowing control of the start of the reaction when the reaction mixture is adequately sparged of oxygen.

In some embodiments, the initiator may also be a redox pair such as persulfate/bisulfate, persulfate/thiosulfate, persulfate/ascorbate, hydrogen peroxide/ascorbate, sulfur dioxide/tert-butylhydroperoxide, persulfate/erythorbate, tert-butylhydroperoxide/erythorbate and/or tert-butylperbenzoate/erythorbate. These initiators are able to initiate the reaction at room temperature, thereby minimizing the chance of heating the reaction mixture to the boiling point of the aqueous phase as heat is removed through the jacket around the reactor. However, homogeneous mixing may not accomplished by the time the reaction is initiated and there may be rapid polymerization of the surface of the droplets with much slower polymerization within the bead.

In preferred embodiments, the reaction is not started immediately after the mixing of the aqueous phase into the oil phase in the final reactor because the aqueous phase still has an excessive amount of oxygen dissolved in the water. It will be appreciated by one of skill in the art that an excessive amount of oxygen may cause poor reactivity and inadequate mixing may prevent the establishment of uniform droplet sizes. Instead, the final reaction mixture is first sparged with the inert gas for ten to sixty minutes after all reagents (except the redox pair if that initiator system is used) have been placed in the reactor. The reaction may be initiated when a low oxygen content (e.g., below 15 ppm) is measured in the inert gas exiting the reactor.

It will be appreciated by those of skill in the art that with acrylate and methacrylate monomers polymerization begins in the droplets and progresses to a point where coalescence of the beads becomes more likely (the “sticky phase”). It may be necessary that a second addition of surfactant (e.g., appropriately degassed to remove oxygen) be added during this phase or that the agitation rate be increased. For persulfate thermal initiation, this sticky phase may occur at about 50 to 55° C. For redox initiation systems, the need for additional surfactant may be lessened by the initial surface polymerization, but if additional surfactant is needed, it should be added as soon as an exotherm is noted.

The reaction may be continued for four to six hours after the peak exotherm is seen to allow for maximal consumption of the monomer into the polymer. Following the reaction, the beads may be isolated by either transferring the entire reaction mixture to a centrifuge or filter to remove the fluids or by initially distilling the water and some of the oil phase (e.g., frequently as an azeotrope) until no further removal of water is possible and the distillation temperature rises significantly above 100° C. followed by isolating the beads by either centrifugation or filtering. The isolated beads are then dried to a residual moisture content (e.g., less than 5%).

An exemplary cross-linked polyelectrolyte, polyacrlylate, may be formed by copolymerizing an ethylenically unsaturated carboxylic acid with a multifunctional cross-linking monomer. The acid monomer or polymer may be substantially or partially neutralized with an alkali metal salt such as the hydroxide, the carbonate, or the bicarbonate and polymerized by the addition of an initiator. One such exemplary polymer gel is a copolymer of acrylic acid/sodium acrylate and any of a variety of cross-linkers.

The reactants for the synthesis of exemplary cross-linked polyelectrolyte polymeric beads, such as cross-linked polyacrylate, are provided in Table 2 below. These cross-linked polyelectrolyte polymeric beads may be produced as a one-hundred kilogram batch in a five-hundred gallon vessel.

TABLE 2
List of Components Used in the Manufacture of Cross-linked
Polyacrylate Beads

| | | Amount/ |
|---|---|---|
| | | batch |
| Component | Function | (kg) |
| Acrylic Acid | Monomer | 88 |
| Water | Solvent | 90 |
| 50% Sodium Hydroxide | Neutralization of acrylic | 79 |
| | acid monomer | |
| Naphtha [petroleum], | Continuous phase for | As needed |
| hydrotreated | suspension | |
| heavy, (Isopar L) | | |
| Fumed silica (Aerosil R972) | Suspending agent | 0.9 |
| Diethylenetriaminepentaacetic | Control of metal ions in | 0.9 |
| Acid | | |
| Pentasodium | reagents, solvents, or | |
| Sodium Persulfate | Polymerization initiator | 0.06 |
| Trimethylolpropane Triacrylate, | Cross-linking agent | 0.3 |
| (TMPTA) | | |

An exemplary polymerization reaction is shown below.

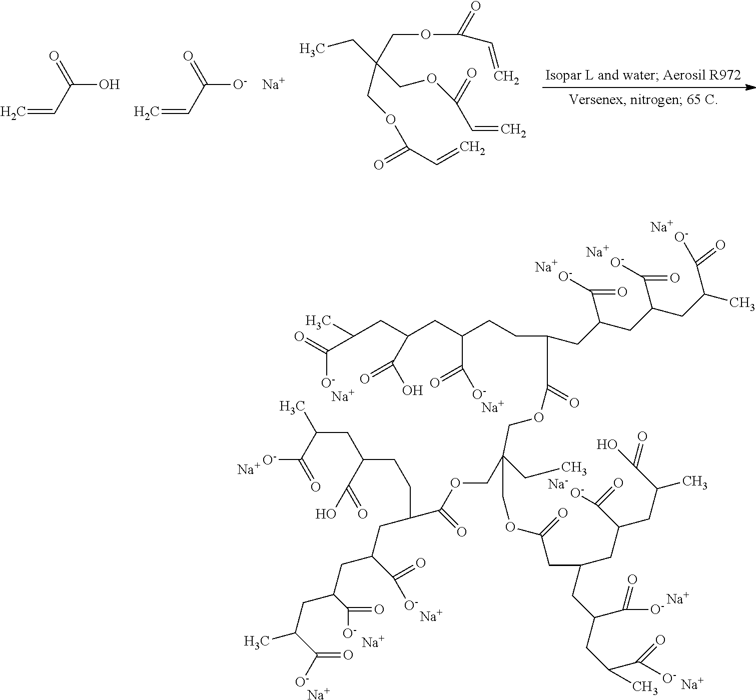

2. Preparation of Cross-Linked Polyelectrolyte Polymeric Beads with Hydrogen Counterions

Partially neutralized or non-neutralized polyelectrolyte polymers may be prepared with 100% hydrogen counterion content by washing the polymer with acid. Suitable acids contemplated for use with the present disclosure, include, for example, hydrochloric acid, acetic acid and phosphoric acid.

Those skilled in the art will recognize that the replacement of the counterions, including cations such as sodium atoms, by hydrogen atoms may be performed with many different acids and different concentrations of acid. However, care must be taken in choice of acid and concentration to avoid damage to the polymer or the cross-linkers. For instance, nitric and sulfuric acids would be avoided.

Acid washed polyelectrolyte polymers may then be dried in a vacuum oven or inert atmosphere until less than 5% moisture remains to produce cross-linked polyacrylic acid which is substantially the free acid form of lightly cross-linked polyacrylic acid. Optionally, if the intact bead form of partially-neutralized, lightly cross-linked polyacrylate is used, the cross-linked polyelectrolyte polymer may be left in the bead form recovered from the oven or may be milled to obtain smaller particles of low-sodium cross-linked polyelectrolyte polymer.

3. Preparation of Cross-Linked Polyelectrolyte Polymeric Beads with Varying Counterion Content

The free acid form of cross-linked polyelectrolyte polymers of the present disclosure, including, for example, cross-linked polyacrylic acid may be converted into polymer with various levels of one or more counterions (e.g., one or more inorganic counterions, such as sodium, potassium, calcium, magnesium and/or ammonium and/or one or more organic counterions, such as choline and/or lysine). These methods may be carried out with intact beads, with disrupted beads, or with powdered forms of cross-linked polyelectrolyte polymers, including for example, polyacrylate polymers.

Suitable counterions include alkali metals and alkaline earth metals, including, for example, sodium, potassium, calcium or magnesium and exclude hydrogen. Counterions may be selected based on the requirements of an individual patient. For example, by appropriate selection of counterions electrolytic imbalances in patients may be treated. For example, in patients having excess sodium, sodium would be avoided as a counterion.

Counterions may be provided as salts that could be dissolved to a sufficient degree in aqueous solution and mixed with the acid form of the polymer. Particularly advantageous choices of salts would be those that neutralize the acid in such a way as to produce products that are easily removed from the polymer. Such salts include the carbonate salt of the desired counterion (e.g. sodium carbonate, potassium carbonate, calcium carbonate), the bicarbonate salt of the desired counterion (e.g. calcium bicarbonate, magnesium bicarbonate, lithium bicarbonate), or the hydroxide or oxide of the desired counterion (e.g. sodium hydroxide, choline hydroxide, magnesium hydroxide, magnesium oxide).

4. Preparation of Cross-Linked Polyelectrolyte Polymeric Particles with Increased Saline Holding Capacity

Partially neutralized or non-neutralized polyelectrolyte polymers of the present disclosure, including cross-linked polyelectrolyte polymeric beads, may be disrupted to increase their saline holding capacity. Saline holding capacity is preferably determined as described in Example 4, wherein the beads or disrupted beads are include with a neutral pH (e.g., pH 7) saline solution having a sodium concentration of 0.15 M. Alternatively, a 0.9% saline solution (0.154 M sodium) may be used.

Cross-linked polyelectrolyte polymeric beads, including cross-linked polyacrylate polymeric beads, may be disrupted into smaller particles, for example, by milling or crushing in a grinder. The disrupted polymeric beads may be washed (e.g., to remove soluble polymer). Suitable washing solutions include purified water such as deionized water or distilled water and various alcohols. Since the polymer is to be dried, it is desirable to use fluids that will evaporate easily without leaving any residue, such as salts, in the dried polymer. Alternatively, cross-linked polyelectrolyte polymeric beads, including cross-linked polyacrylate polymeric beads, may be disrupted by placing the beads into purified water or other sutiable solvents and agitating the beads (e.g., stirring with a magnetic stir bar or agitating at 500 rpm overnight), so that the residual soluble polymer in the polymeric beads may be reduced or eliminated, the beads may be disrupted and the saline holding capacity of the polymeric beads increased.

Particles of a certain size, may be obtained by sieving through sieves such as screens. Screens may be stacked to obtain particles with a range of sizes. Screens are shaken to allow particles to sift through and get caught on the screen with an opening just below their diameter. For example, particles that pass through an 18 Mesh screen and are caught on a 20 Mesh screen are between 850 and 1000 microns in diameter. Screen mesh and the corresponding particle size allowed to pass through the mesh include, 18 mesh, 1000 microns; 20 mesh, 850 microns; 25 mesh, 710 microns; 30 mesh, 600 microns; 35 mesh, 500 microns, 40 mesh, 425 microns; 45 mesh, 35 microns; 50 mesh, 300 microns; 60 mesh, 250 microns; 70 mesh, 212 microns; 80 mesh, 180 microns; 100 mesh, 150 microns; 120 mesh, 125 microns; 140 mesh, 106 microns; 170 mesh, 90 microns; 200 mesh, 75 microns; 230 mesh, 63 microns; and 270 mesh, 53 microns. Thus particles of varying sizes may be obtained through the use of one or more screens.

### Therapeutic Uses

The disclosed polymers have a variety of uses, including therapeutic uses. Such uses may include methods for the removal of fluid. Such uses may also include methods for treating diseases or disorders associated with increased retention of fluid and/or ion imbalances. The disclosed polymers may be used in methods to treat end stage renal disease (ESRD), chronic kidney disease (CKD), congestive heart failure (CHF) or hypertension. The disclosed polymers may also be used in methods to treat an intestinal disorder, a nutritional disorder (e.g., kwashiorkor or gluten-sensitive enteropathy), a hepatic disease (e.g., cirrhosis of the liver), an endocrine disorder (e.g., preclampsia or eclampsia), a neurological disorder (e.g., angioneurotic edema) or immune system disorder. The discloses polymers may be administered in combination with agents that increase fluid in the intestine (e.g., osmotic agents, irritants, sodium absorption blocking agents and agents that enhance fluid secretion).

In some embodiments, the absorbent material may be encapsulated in a capsule. The capsules may be coated with a coating that allows it to pass through the gut and open in the intestine where the material may absorb fluid or specific ions that are concentrated in that particular position of the intestine. The individual particles or groups of particles may be encapsulated or alternatively, larger quantities of beads or particles may be encapsulated together.

In an exemplary method, the swelling rate of the polymer may be controlled by selecting particle or bead size, and or polymer with varied level of ion loading, to provide delivery of the polymer to specific locations in the gut before extensive swelling occurs. Larger sized particles have slower swelling rates. When given orally, the absorbent material may be used to supplement or replace dialysis treatments in dialysis patients, to supplement or replace diuretic therapy in patients with congestive heart failure, to supplement or replace diuretic and antihypertensive therapy in patients with hypertension and to supplement or replace these and dietary measures for treatment of fluid and/or sodium overload and/or potassium overload in patients with other diseases and syndromes, including those causing fluid retention in the body.

The methods may be used to modulate (e.g., increase or decrease) levels of one or more ions, including more than one ion, in a subject by administering a composition of the present disclosure to the subject in an amount effective to modulate the levels of one or more ions, including more than one ion, in the subject.

The composition may bind to one or more ions in the subject thereby decreasing the levels of one or more ions in the subject. Additionally, the composition may release one or more ions in the subject thereby increasing the levels of one or more ions in the subject. Alternatively, the composition may bind to one or more first ions in the subject thereby decreasing the levels of one or more first ions in the subject and the composition release one or more second ions in the subject thereby increasing the levels of one or more second ions in the subject.

The composition may be used to remove one or more ions selected from the group consisting of: hydrogen, sodium, potassium, calcium, magnesium and/or ammonium.

### Pharmaceutical Compositions

Pharmaceutical compositions are disclosed comprising a cross-linked polyelectrolyte polymer, including cross-linked polyelectrolyte polymeric beads, of the present disclosure. These compositions may be delivered to a subject, including a subject using a wide variety of routes or modes of administration. Preferred routes for administration are oral or intestinal.

A pharmaceutical composition or dosage form, including wherein the polymer is in admixture or mixture with one or more pharmaceutically acceptable carriers, excipients or diluents. Pharmaceutical compositions for use in accordance with the present disclosure may be formulated in conventional manner using one or more physiologically acceptable carriers compromising excipients and auxiliaries which facilitate processing of the polymer into preparations which may be used pharmaceutically. Proper formulation is dependent upon the route of administration chosen. Such compositions may contain a therapeutically effective amount of polymer and may include a pharmaceutically acceptable carrier. Pharmaceutically acceptable carriers include those approved by a regulatory agency of the Federal or a state government or listed in the U.S. Pharmacopeia or other generally recognized pharmacopeia for use in animals, and more particularly, in humans. Carriers can include an active ingredient in which the disclosed compositions are administered.

For oral administration, the disclosed compositions may be formulated readily by combining them with pharmaceutically acceptable carriers well known in the art. Such carriers enable the compositions of the disclosure to be formulated, preferably in capsules but alternatively in other dosage forms such as tablets, pills, dragees, capsules, liquids, gels, syrups, slurries, suspensions, wafers, sachets, powders, dissolving tablets and the like, for oral ingestion by a subject, including a subject to be treated. In some embodiments, the compositions or capsules containing the compositions, do not have an enteric coating.

The amount of the active cross-linked polyelectrolyte polymer, including cross-linked polyelectrolyte polymeric beads, are present in an effective amount, including, for example, in an amount effective to achieve therapeutic and/or prophylactic benefit. Effective doses may be extrapolated from dose-response curves derived from in vitro or animal model test systems. Dosage amount and interval may be adjusted individually to provide levels of cross-linked polyelectrolyte polymer, including cross-linked polyelectrolyte polymeric beads that are sufficient to maintain the desired therapeutic effect. The dosage regimen involved in a method of treatment may be determined by the attending physician, considering various factors which modify the action of polymer, e.g. the age, condition, body weight, sex and diet of the subject, the severity of disease, time of administration and other clinical factors.

The amount of compound administered will, of course, be dependent on the subject being treated, on the subject's weight, the nature and severity of the affliction, the manner of administration, and the judgment of the prescribing physician. The therapy may be repeated intermittently while symptoms are detectable or even when they are not detectable. The therapy may be provided alone or in combination with other agents.

The polyelectrolyte polymer of the present disclosure may be administered in combination with other therapeutic agents. The choice of therapeutic agents that may be co-administered with the compositions of the disclosure will depend, in part, on the condition being treated.

### EXAMPLES

### Example 1

This example demonstrates the preparation of an exemplary cross-linked polyelectrolyte polymer, such as a lightly crosslinked polyacrylic acid partially neutralized with sodium.

An inverse suspension process may be used with the following components: a monomer (e.g., polyacrylic acid), solvent (e.g., water), base for neutralization of monomer (e.g., NaOH), lipophilic solvent (e.g., Isopar L), suspending agent (e.g., fumed silica such as Aerosil R972), chelating agent (e.g., Versenex-80), polymerization initiator (e.g., sodium persulfate), and cross-linking agent (e.g., TMPTA). For example, cross-linked polyacrylate beads were prepared by adding eighty-eight kilograms acrylic acid and about eighty-seven kilograms of water to a suitable, agitated vessel and sparging air through the mixture. The mixture was continuously agitated and cooled while seventy-nine kilograms of 50% sodium hydroxide was added while the temperature of the mixture was advantageously maintained below about 40° C. In this manner about 80% neutralization of the acrylic acid was obtained. If desired, neutralization percentages of from about 60% to 100% were obtained by altering the amount of sodium hydroxide. Alternatively other basic sodium salts, such as sodium carbonate or sodium bicarbonate, are used in addition to basic salts of other alkali metals.

To a second, suitable, agitated reactor, about seven-hundred kilograms of Isopar L (or other lipophilic solvents such as toluene, heptane, cyclohexane) was added to 0.3 kilograms of fumed silica (Aerosil R972) that is pre-dispersed in about twenty kilograms of Isopar L (or other lipophilic solvent). Next, about 0.9 kilograms of Versenex-80 solution was added to the partially neutralized acrylic acid solution followed by the addition of 0.3 kilograms of trimethylolpropane triacrylate to the Isopar L/Aerosil R972 dispersion. About 0.06 kilograms sodium persulfate as a solution in about three kilograms of water was added to the partially neutralized acrylic acid solution. The partially neutralized acrylic acid solution may then be filtered.

The partially neutralized acrylic acid solution was transferred into the Isopar L in the second reactor. Optionally, the partially neutralized acrylic acid solution may be filtered at this point. The mixture was agitated for about fifteen to thirty minutes to achieve suspension of the aqueous monomer droplets while nitrogen (or other suitable inert gas) was sparged through the mixture during the agitation period. The reactor temperature may be increased to about 50° C. at which point a second dispersion of Aerosil R972 (0.6 kilograms of Aerosil 8972 in about twenty kilograms of Isopar L) may be added to the reaction mixture. Polymerization of the mixture was completed by heating the reaction mixture to about 65° C. and holding the contents at about 65° C. for about two to four hours after the peak exotherm was observed. The reactor contents were then cooled and placed under vacuum to remove water. About two-hundred and twenty kilograms of distillate was collected. The beads were isolated by centrifugation and dried under vacuum with a nitrogen bleed, if needed, at about 100° C.

The beads were screened to remove oversized agglomerates and fines. Typically, about one-hundred kilograms of cross-linked polyacrylate beads were obtained. If the residual acrylic acid level is too high, the cross-linked polyacrylate beads are reloaded to a suitable reactor containing Isopar L, water, and a small amount of sodium persulfate. After sparging the mixture with nitrogen, the beads were incubated at about 70° C. for about two to three hours. The mixture was then cooled and the cross-linked polyacrylate beads isolated, dried, and screened as before.

When the beads were screened, the mean particle size for the beads generally ranged from about 700 microns to about 1200 microns. The upper screen size ranged from 840 to 1400 microns (e.g., 24-16 mesh) and the lower screen size ranged from 540 to 840 microns (e.g., 36-24 mesh).

Optionally, the beads are placed into capsules (e.g., hard size 00 HPMC capsules). Such capsules are optionally coated. The following materials are used to prepare an exemplary coating suspension (% w/w): Eudragite L30D-55 (53.76%), Plasacryl (6.45%), triethyl citrate (2.58%) and sterile water (37.20%). For example, L30D-55 is dispensed into a steel container with agitation to create a vortex. Next, sterile water, Plasacryl and triehtyl citrate are added to the vortex. The capsules may then be sprayed with the mixture followed by drying.

### Example 2

This example demonstrates the preparation of an exemplary cross-linked polyelectrolyte polymer, such as a cross-linked polyacrylate polymer.

Cross-linked polyelectrolyte was prepared on a smaller scale by placing 14.7 kg Isopar L (or other inert hydrocarbon solvent such as toluene, cyclohexane, or n-heptane) into a jacketed, thirty liter glass or stainless steel reactor fitted with two low-shear, high-viscosity impellers and two baffles. 0.0086 kg of fumed silica, such as, Aerosil 8972 and 0.5 kg of Isopar L (or whichever hydrocarbon solvent has been chosen) were added to the high shear blender such as a Waring blender to disperse the Aerosil into the solvent for two minutes. Next, the mixture was added to the thirty liter reactor. The solution was then agitated in the thirty liter reactor while an inert gas was sparged through the room temperature solution.

A second batch of 0.5 kg Isopar L (or whichever hydrocarbon solvent has been chosen) with 0.0086 kg Aerosil R972 was prepared in a high shear blender. This suspension was placed into a vessel and an inert gas (nitrogen, argon, etc) sparged through it to degas it. The degassing was continued until the solution as used.

About 1.72 kg glacial acrylic acid and 1.72 kg water as placed into a twelve liter jacketed reactor and the temperature lowered to about 15° C. With vigorous stirring, 1.53 kg of 50% NaOH solution was added while keeping the temperature below 30° C. Air may be maintained in the reaction mixture by bubbling through the solution, if needed. When the neutralization addition was completed, 0.069 kg of 10% Versenex 80 solution was added to the reactor and mixed. After a few minutes, 0.009 kg of freshly prepared 10% sodium persulfate solution was added to the reactor and mixed for a few minutes. The solution was then transferred to the thirty liter reactor.

About 0.006 kg of trimethylolpropane was added to the thirty liter reactor. The agitation was continued in the thirty liter reactor while de-gassing by bubbling an inert gas through the mixture for 40 to 60 minutes. The solution was kept at room temperature. After the 40 to 60 minutes of degassing, the temperature of the reaction mixture was quickly raised by circulation of a 90 to 95° C. solution through the jacket of the jacketed reactor while continuing the degassing and agitation. When the reaction mixture reaches 50° C., the second batch of Aerosil R972 was rapidly added. When the reaction mixture reaches 60° C., the temperature of the heating bath was reduced to 65° C. and the reaction mixture maintained at 65° C. for 2 to 4 hours.

After two to four hours, the reaction mixture was distilled under partial vacuum until no water is being removed and the reaction mixture is cooled to room temperature. The beads were filtered from the liquid and dried under an inert atmosphere until less than 5% moisture remains. Alternatively, the beads are isolated by filtration immediately after the two to four hours of reaction time, rinsed with the organic solvent, and dried under an inert atmosphere. These beads were then processed in the same manners mentioned above to disrupt the beads and washed with purified water to produce the high saline holding capacity CLP described.

### Example 3

This example demonstrates the preparation of an exemplary cross-linked polyelectrolyte polymer, such as a cross-linked polyacrylate polymer.

The bead form of lightly cross-linked, 80% neutralized polyacrylic acid was prepared in a 500 gallon reactor by loading 1775.5 pounds of Isopar L into the reactor and adding 0.4 pounds of Aerosil R972 which had been mixed with high shear in 50.5 pounds of Isopar L. Agitation and nitrogen purge at 500 scfh was started. In a separate reactor, 1953 pounds of acrylic acid was mixed with 20.7 pounds of water and sparged with air. 176.5 pounds of 50% NaOH solution were added to the acrylic acid over 1.25 hours while the temperature was maintained below 40° C. To this solution, 2.0 pounds of Versenex 80 solution, 0.71 pounds trimethylolpropane triacrylate, and 0.158 pounds of sodium persulfate were added. This solution was then transferred to the primary reactor with continued sparging. A second Aerosil charge was prepared using 1.3 pounds of Aerosil in 50.9 pounds of Isopar L with high shear agitation. After approximately 1 hour of sparging, the reactor was heated to a maximum of 78° C. and held in the heated state for 4 to 5 hours. The reactor was then placed under vacuum and distillation was performed for about 5 hours. The remaining reaction mixture was transferred to a centrifuge where the beads are separated and moved to a drier. The dried beads were sieved to select for beads between 710 microns and 1000 microns.

### Example 4

This example describes an exemplary method for determining saline holding capacity of a cross-linked polyelectrolyte polymer, such as a cross-linked polyacrylate polymer.

A pH seven buffer of sodium phosphate tribasic (Na₃PO₄.12H₂0; MW 380.124) was prepared by dissolving 19.0062 grams in about 950 milliliters pure water and adjusting the pH to a final pH of seven±0.1 with 1N HCl before final dilution to one liter resulting in a solution with a sodium concentration of 0.15 M. Next, an amount of cross-linked polyelectrolyte, for example, cross-linked polyacrylate beads (e.g., 0.2±0.05 grams), were transferred to a tared tube and the mass of the beads recorded as in W1. Next, the tube was returned to the balance to record the weight of the tube plus the sample as W2. An excess (e.g., more than seventy times the mass of polymer) amount of the pH 7.0 buffer (e.g., ten milliliters) was then transferred to the tube containing the CLP sample. The tube was then placed on a flat bed shaker with shaking for two, four or six hours. When reduced sodium cross-linked polyacrylate polymer was being tested for saline holding capacity, this time may be extended to twenty-four hours. After shaking, all excess fluid was removed from the tube (e.g., no visible fluid in the tube). Last, the tube and sample were weighed and recorded as W3. The saline holding capacity (SHC) was calculated by dividing the mass of the dry cross-linked polyacrylate beads into the mass of the fluid absorbed, for example, SHC (g/g)=(W3-W2)/ (W1). According to the present disclosure, cross-linked polyelectrolyte polymeric beads, including polyacrylate beads prepared as described in Example 1, have a saline holding capacity of twenty grams per gram, forty grams per gram or more. Alternatively stated, such cross-linked polyelectrolyte polymeric beads, including where the polyelectrolyte is polyacrylate, may absorb 20-fold, 40-fold, or more of their mass in a saline solution.

### Example 5

This example demonstrates the preparation of cross-linked polyelectrolyte polymers, such as cross-linked polyacrylate polymers, with a high saline holding capacity.

In an exemplary method, beads prepared according to Example 1 (Lot MM 050623-B sieved to 710 to 1000 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. A one gram sample of beads was then placed into 1100 milliliters of distilled water and stirred at 500 rpm for sixteen hours. The beads swelled in the distilled water and were disrupted by the vigorous stirring. Next, the solution was filtered and revealed 0.4 grams of soluble polymer in this filtrate. The isolated disrupted beads were washed with another 1000 milliliters of distilled water. The disrupted beads were then dried in a vacuum oven at about 100° C. until no further moisture could be removed. The dried, disrupted, washed beads were then tested for saline holding capacity and were found to absorb 92 grams of neutral 0.154 M saline per gram of material after four hours and 110 grams of neutral 0.154 M saline after sixteen to twenty-four hours.

### Example 6

This example demonstrates the preparation of cross-linked polyelectrolyte polymers, such as cross-linked polyacrylate polymers, with a high saline holding capacity.

In an exemplary method, beads prepared according to Example 1 (Lot MM 050623-B sieved to 710 to 1000 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. A one gram sample of beads was then placed into a mechanical mill and milled for three bursts of ten seconds per burst. The resulting particles of disrupted beads were placed into 1000 milliliters of distilled water and stirred at 500 rpm overnight. Evaporation of this filtrate revealed that 0.33 grams of soluble polymer was removed from the crushed, washed beads. The water was then removed by filtration and the disrupted beads are washed with another 1000 milliliters of distilled water. The disrupted beads were then dried in a vacuum oven at about 100° C. until no further moisture could be removed. The disrupted beads were tested for absorption of neutral 0.154 M saline and found to absorb 90 grams of saline per gram of polymer after four hours and 112 grams of saline per gram of polymer after sixteen to twenty-four hours.

### Example 7

This example demonstrates the disruption of cross-linked polyelectrolyte polymers, such as cross-linked polyacrylate polymers, by milling.

In an exemplary method, beads prepared according to Example 1 were milled to produce cross-linked polyacrylate particles, for example, a grinding apparatus (e.g., a COMIL® apparatus) was loaded with the polyacrylate beads to just below the top of the impeller blade. The impeller was then turned on and set to 100% power. The grinding apparatus was stopped every thirty minutes and allowed to cool for ten minutes before milling is resumed. Next, the milled material was poured through a sieving apparatus (e.g., a VORTI-SIV® apparatus) set up with two screens (e.g., US Mesh # 35 and US Mesh # 70) to collect polyacrylate particles that are from 212 to 500 microns. Material greater than 500 microns was collected and again milled with the resulting particles again sieved for those particles between 212 to 500 microns. Milling and sieving may continue until the material greater than 500 microns no longer reduces in particle size. Particles less than 212 microns were collected through the grinding and sieving process as powder for use or may be discarded. The particles that were 212 to 500 microns were tested for saline holding capacity which was determined to be approximately 54 grams per gram. The particles that were 212 to 500 microns were encapsulated in a capsule. These capsules were coated with a pH 5.5 release enteric coating and were tested for saline holding capacity which was determined to be ≧70 grams per gram.

### Example 8

This example demonstrates the preparation of cross-linked polyelectrolyte polymers, such as cross-linked polyacrylate polymers, with a high saline holding capacity.

In an exemplary method, beads prepared according to Example 1 (Lot MM 050902-B sieved to 710 to 1000 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. The beads were then disrupted as in Example 5. Saline holding capacity of the crushed, washed, and dried polymer was measured as 72 grams per gram at four hours and 103 grams per gram at eight hours.

### Example 9

This example demonstrates the preparation of cross-linked polyelectrolyte polymers, such as cross-linked polyacrylate polymers, with a high saline holding capacity.

In an exemplary method, beads prepared according to Example 1 (Lot MM 050922-A sieved to 710 to 1000 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. The beads were then disrupted as in Example 5. Saline holding capacity of the crushed washed, and dried polymer was measured as 85 grams per gram at four hours.

### Example 10

This example demonstrates the preparation of cross-linked polyelectrolyte polymers, such as cross-linked polyacrylate polymers, with a high saline holding capacity.

In an exemplary method, beads prepared according to Example 1 (Lot MM 050624 sieved to 710 to 1000 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. The beads were then disrupted as in Example 5. Saline holding capacity of the crushed, washed, and dried polymer was measured as 71 grams per gram at four hours and 84 grams per gram at eight hours.

### Example 11

This example demonstrates the preparation of cross-linked polyelectrolyte polymers, such as cross-linked polyacrylate polymers, with a high saline holding capacity.

In an exemplary method, beads prepared according to Example 1 (Lot MM 050923-D sieved to 500 to 710 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. The beads were then disrupted as in Example 5. Saline holding capacity of the crushed, washed, and dried polymer was measured as 72 grams per gram at four hours and 86 grams per gram at twenty hours.

### Example 12

This example demonstrates the preparation of cross-linked polyelectrolyte polymers, such as cross-linked polyacrylate polymers, with a high saline holding capacity.

In an exemplary method, beads prepared according to Example 1 (Lot MM 050927 sieved to 500 to 710 micron diameter) were tested for saline holding capacity by measuring the amount of neutral 0.154 M saline absorbed by three samples of approximately 0.2 grams of the beads over four hours. The saline holding capacity was approximately 60 grams saline per gram of beads at four hours and remained constant at this value after sixteen to twenty-four hours. The beads were then disrupted as in Example 5. Saline holding capacity of the crushed, washed, and dried polymer was measured as 90 grams per gram at four hours and 97 grams per gram at twenty hours.

### Example 13

This counter-example demonstrates the failure of preparation of a high saline holding capacity lightly crosslinked, partially neutralized polyacrylic from an aqueous polymerization process.

Crosslinked 80% sodium neutralized polyacrylate particles prepared by aqueous polymerization, drying, and crushing (Lot 00612DH, Sigma-Aldrich) were tested for saline holding capacity by measuring the amount of neutral 0.154 M saline absorbed by three samples of approximately 0.2 grams of the particles over four hours. The saline holding capacity was approximately 46 grams saline per gram of particles at four hours and 45 grams saline per gram polymer at twenty hours. The particles were then disrupted as in Example 5. Saline holding capacity of the crushed, washed, and dried polymer particles was measured as 48 grams per gram at four hours and 52 grams per gram at twenty hours.

While the present disclosure has been described and illustrated herein by references to various specific materials, procedures and examples, it is understood that the disclosure is not restricted to the particular combinations of material and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A cross-linked polyelectrolyte polymer that absorbs about 60-fold or more of its mass in aqueous saline.

2. The cross-linked polyelectrolyte polymer of claim 1, wherein the polymer is substantially free of soluble polyacrylic acid polymer.

3. The cross-linked polyelectrolyte polymer of claim 1, wherein the polymer absorbs more than 60-fold or more of its mass in aqueous saline.

4-8. (canceled)

9. The cross-linked polyelectrolyte polymer of claim 1, wherein the polymer further comprises counterions.

10. The cross-linked polyelectrolyte polymer of claim 9, wherein the counterions comprise cations.

11. The composition of claim 1, wherein the polymer comprises one or more bound inorganic counterions.

12. The composition of claim 11, wherein the inorganic counterion is selected from the group consisting of: hydrogen, sodium, potassium, calcium, magnesium and ammonium.

13. The composition of claim 1, wherein the polymer comprises one or more bound organic counterions.

14. The composition of claim 13, wherein the organic counterion is selected from the group consisting of: lysine, choline and arginine.

15. The composition of claim 1, wherein the cross-linked polyelectrolyte polymeric beads comprises one or more inorganic counterions and at least one or more organic counterions.

16. The cross-linked polyelectrolyte polymer of claim 1, wherein the polymer is substantially in the shape of a disrupted sphere or ellipsoid.

17. The composition of claim 16, wherein the disrupted sphere or ellipsoid has a particle size of about 210 to 500 microns.

18. The cross-linked polyelectrolyte polymer of claim 1, wherein the polymer is substantially in the shape of a disrupted sphere or ellipsoid and the polymer is substantially free of soluble polyacrylic acid polymer.

19. The cross-linked polyelectrolyte polymer of claim 1, wherein the polymer is substantially coated with a coating.

20. The composition of claim 1, wherein the polymer is polyacrylate.

21. A pharmaceutical composition comprising the composition of claim 1.

22. A method of removing fluid from a subject comprising administering the cross-linked polyelectrolyte polymer of claim 1 to the subject in an amount effective to remove fluid.

23-42. (canceled)

43. A method of removing one or more waste products from a subject comprising administering a cross-linked polyelectrolyte polymer of claim 1 to the subject in an amount effective to remove an amount of one or more waste products from the subject.

44-52. (canceled)

53. A method of preparing cross-linked polyelectrolyte polymeric particles capable of absorbing greater than 60 times their mass of an aqueous saline solution comprising:
a) obtaining a cross-linked polyelectrolyte polymer in a spherical or nearly spherical form;
b) disrupting the polymer into particles; and
c) washing the particles.

54-70. (canceled)

71. A cross-linked polyelectrolyte particle prepared by claim 53.
